(19) United States
(12) Patent Application Publication
(10) Pub. No.: US 2016/0158344 A1
(43) Pub. Date: Jun. 9, 2016

(54) Methods And Compositions of Protein Antigens For The Diagnosis And Treatment of Herpes Simplex Viruses Type 1 And 2

(71) Applicant: ImmPORT Therapeutics, Inc., Irvine, CA (US)

(72) Inventors: David Huw Davies, Long Beach, CA (US); Xiaowu Liang, La Jolla, CA (US); Philip Felgner, Rancho Santa Fe, CA (US)

(21) Appl. No.: 15/019,864

(22) Filed: Feb. 9, 2016

Related U.S. Application Data
(62) Division of application No. 14/130,472, filed on Jun. 27, 2014.
(60) Provisional application No. 61/503,790, filed on Jul. 1, 2011.

(51) Int. Cl. A61K 39/245; G01N 33/549; C12N 7/00

(52) U.S. Cl. CPC A61K 39/245; C12N 7/00; G01N 33/549; C12N 2710/16634; A61K 2039/70

(57) ABSTRACT

Contemplated compositions, devices, and methods are drawn to various antigens from Herpes Simplex Virus type 1 (HSV-1) and Herpes Simplex Virus type 2 (HSV-2) and their use in vaccines, therapeutic agents, and various diagnostic tests. In particularly preferred aspects, the antigens are immunodominant and have quantified and known relative reactivities with respect to sera of a population infected with the pathogen, and/or have a known association with a disease parameter.

This application is a divisional application of co-pending U.S. application Ser. No. 14/130,472, filed Dec. 31, 2013, which is a US national phase filing of International Application No. PCT/US12/44814, which was filed Jun. 29, 2012 which claims priority to our U.S. provisional application with the Ser. No. 61/503,790, which was filed Jul. 1, 2011, all of which are incorporated by reference herein in their entirety.

### FIELD OF THE INVENTION

The field of the invention is compositions and methods related to selected antigens of Herpes Simplex, especially as they relate to their use in diagnosis, treatment, and therapeutic compositions.

### BACKGROUND

Herpes Simplex Virus type 1 (HSV-1) and Herpes Simplex Virus type 2 (HSV-2) are viri capable of infecting both mammals and birds. HSV type 1 and 2 are significant causes of human morbidity. HSV-2 is sexually transmitted and is the causative agent of most recurrent genital herpes lesions. Infection with HSV-2 is associated with increased pregnancy risks that include spontaneous abortion, premature birth, and congenital infection of the newborn with the virus. In addition, infection with HSV-2 is also associated with an increased risk of infection when exposed to HIV. Unfortunately HSV-2 infections are often asymptomatic and most infected individuals are unaware they are infected. This ignorance of HSV-2 status is a major contributing factor to transmission to uninfected partners. In contrast, HSV-1 is usually transmitted during childhood, and is predominantly associated with orolabial infections, which result in “cold sores”. HSV-1 can, however cause corneal infection that can lead to blindness. Unfortunately, this differing distribution of lesions is insufficient to distinguish between HSV-1 and HSV-2.

Both HSV-1 and HSV-2 establish lifelong latent infections in their hosts. These infections are characterized by periodic reactivation of the infection and resultant virus shedding. Due to the different natural histories and outcomes of HSV-1 and HSV-2 infections, accurate diagnosis of the HSV type is important for patient management and prognosis, and for controlling potential transmission. For example, knowing the specific HSV type can help the patient take appropriate precautions to prevent transmission of the disease to others. In particular, the identification of unrecognized HSV-2 infection can be used to carefully monitor viral shedding during pregnancy and thereby minimize the risk of congenital infection.

As a further complication to HSV typing, HSV-1 and HSV-2 show close amino acid sequence homology and therefore exhibit extensive antigenic cross-reactivity. A few antigens are sufficiently divergent that they allow discrimination of HSV-1 and HSV-2 infection, although only the US4/glycoprotein G (gG) of HSV-1 and -2 are sufficiently divergent to provide useful sensitivity and specificity parameters. Current diagnostic methods, include enzyme-linked immunoassay (ELISA), fluorescence immunoassay (FIA), and similar testing formats. One such test is described in U.S. Pat. No. 6,265,176, in which patient antibody binding to an immobilized HSV antigen is characterized. Such tests generally characterize the patient's immune response to the virus, and so often utilize recombinant antigens from HSV-1 and HSV-2. For example, EP 1 982 993 A2 describes the use of multimeric fusion peptides that incorporate portions of glycoprotein G for detection of HSV-1 and HSV-2. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply. Currently, the only tests approved by the FDA are based upon gG-1 and gG-2 reactivity. Ambiguous results are common when such tests are based on the use of a single diagnostic antigen, as different infected patients may have varying levels of immune response. Although the gG-based tests show excellent specificity, as with any single-antigen test, there is a risk of obtaining false negative results from infected individuals that simply lack antibodies to gG. As a result follow-up testing, such as whole virus Western blotting, may be required for an accurate diagnosis. Unfortunately, current testing methods yield only partial useful results. Test performance on such partially characterized platforms differs widely, and cross reactivity can easily generate false positive results from such complex mixtures.

In response to the high prevalence and potential negative consequences of HIV infection, attempts have been made to develop vaccines. Such development requires identification of antigens that are not only specific for HSV, but that are also antigenic and capable of generating a protective immune response. For example U.S. Pat. App. No. 2004/241182A1 disclose HSV-2 proteins identified as antigenic and screened for T-cell activation for use in vaccines. Similarly, EP 2 272 859 A2 describes the use of HSV-2 peptides with adjuvants that are selected to induce a Th1-type immune response. A different approach is described in WO 2004/026265A2, which utilized genetic constructs that result in the expression of peptides when introduced into an animal. Such genetic constructs were generated from a library of HSV-1 sequences in order to induce an immune response. Subsequent challenge of the animals with HSV-1 was used to identify constructs that generated a protective immune response. While such an approach may have at least some utility in identifying immunogenic and protective antigens, it is, however, necessarily limited to the production of peptides and proteins that have undergone eukaryotic processing, for example glycosylation and chaperonin-assisted folding. Such peptides and proteins may be difficult to produce at a large scale, as such processes generally involve chemical synthesis or the use of readily cultured genetically modified bacteria.

Consequently, there remains a large, unmet need to provide improved compositions and methods of antigen and antibody detection and monitoring for diagnostic and therapeutic applications related to HSV.

### SUMMARY OF THE INVENTION

In addressing the need for improved compositions and methods of antigen and antibody detection and monitoring for diagnostic and therapeutic applications related to HSV, the instant inventors utilized a proteome-microarray approach to profile the antibody response during infection against multiple HSV proteins in order to identify antigens that generate antibodies with high avidity and specificity that may have more precise serodiagnostic and therapeutic utility.

Proteome microarrays were produced that displayed each of the proteins encoded by HSV-1 and HSV-2 as expressed in E. coli-based in vitro transcription/translation of open reading frames (ORFs). Microarrays thus produced were screened with sera from a panel of patients with ocular and/or genital herpes infections that had been serotyped using commercial gG-1 and gG-2 based HSV serotyping kits. Surprisingly this analysis demonstrated that while HSV-1 seropositive donors reacted well to HSV-1 antigens on the array and demonstrated minimal activity with HSV-2 antigens, HSV-2 seropositive donors reacted well to both HSV-2 and HSV-1 antigens. Thus, while there were several HSV-2 antigens that were recognized specifically by HSV-2 seropositive donors, only HSV-1 US8 (glycoprotein E) was specifically recognized by HSV-1 seropositive donors. A multiplex proteomics approach to characterize infected individual's antibody response to HSV was discovered in which highly sensitive antigens were used in conjunction with highly specific antigens. To the inventors' knowledge, such a multiplexed approach to characterization of the response to HSV infection has not been previously attempted. Utilizing multiplex testing disclosed herein the inventors discovered specific and cross-reactive antigen(s) that provide improved sensitivity and specificity over current testing methods, which rely only on type specific antigens and can produce a high rate of false negatives. In addition, the multiplex testing disclosed herein can diagnose HSV-1 and HSV-2 in a single test, as opposed to current testing methods which require separate tests for HSV-1 and HSV-2.

These results therefore provide for improved diagnosis of HSV related infection(s), and further provide clear, distinct, antigen targets for serodiagnostic, biomarker, vaccine, and therapeutic product development against HSV and the diseases and disorders triggered by HSV in mammals, birds, and humans. In addition the antigens may be expressed in E. coli, simplifying their production on a large scale.

Thus the instant inventive subject matter provides a new and useful tool that can accurately survey HSV-induced diseases. More specifically, the present inventive subject matter provides tools, methods, and compositions for the identification, analysis, and monitoring of specific HSV antigens, or sets of antigens, that have diagnostic, prognostic, and therapeutic value, specifically with respect to various mammalian, bird, and human diseases.

The instant inventive subject matter can be used to identify biologically relevant antigens, sets of antigens, antibodies, and sets of antibodies related to HSV and HSV-related infections and diseases. The inventive subject matter can also enable the monitoring and analysis of treatment efficacy, via longitudinal monitoring of reactivity of an antibody, or a set of antibodies, against select HSV antigens. The present inventive subject matter also provides for the detection of antibody reactivity to specific HSV protein antigens, or antigen sets, which is important in the diagnosis and treatment of HSV-triggered diseases and disorders that include, but are not limited to, genital herpes lesions, spontaneous abortion, premature birth, congenital herpes, orolabial infections (cold sores), herpetic whitlow, and herpes-induced blindness. Contemplated compositions, devices, and methods comprise antibody reactive antigens from HSV that can be used as a vaccine, as diagnostic markers, and as therapeutic agents. In particularly preferred aspects, the HSV antigens have quantified and known relative reactivities with respect to sera of a population infected with HSV, and have a known association with a disease parameter.

Thus, the present inventive subject matter provides for the identification, analysis, and monitoring of antibodies to specific HSV antigens, or antigen sets, which is important in the diagnosis and/or treatment of various HSV-triggered disorders and diseases. The inventive subject matter also provides tools and methods to accurately survey HSV infection and diseases via the combination of: antibody detection and monitoring, and characterized sera samples, especially as they relate to their use in diagnostic and therapeutic compositions and methods.

An antigen composition of the inventive concept may include two or more antigens that are associated with a carrier, where at least two of the antigens have quantified and known relative reactivities with antibodies from sera of individuals affected by HSV-1, and where at least two of the antigens have an association with a disease parameter. Such antigens may be selected from US3, US6, US8, US9, UL7, UL20, UL22, UL36, and UL44, and/or from fragments of these proteins/peptides. In some embodiments of the inventive concept antibodies to least two of the antigens of such a composition may be present in a high percentage of a population exposed to the antigens, for example about 40% or more. In such an embodiment the average binding affinity and/or the average quantity of the antibodies may be in the upper tertile of binding affinity and/or quantity of antibodies produced by a patient. Known relative reactivities can be avidity or strength of reactivity with an antibody and/or activity state of a disease. A disease parameter may be exposure state (e.g., current or previous exposure) to a pathogen and/or an HSV (such as HSV-1), state of the infection (e.g., acute, latent, or recurrent) with a pathogen and/or an HSV (such as HSV-1), and immunity state (e.g., none, partial, or complete).

In another embodiment of the inventive concept, an antigen composition may include two or more antigens that are associated with a carrier, where at least two of the antigens have quantified and known relative reactivities with antibodies from sera of individuals affected by HSV-2, and where at least two of the antigens have an association with a disease parameter. Such antigens may be selected from UL1, UL3, UL5, UL6, UL7, UL10, UL14, UL17, UL18, UL23, UL26, UL26.5, UL27, UL28, UL32, UL34, UL41, UL42, UL44, UL45, UL49, UL50, UL51, UL54, US6, US7, US8, US9 and US11, and/or from fragments of these proteins/peptides. In some embodiments of the inventive concept antibodies to least two of the antigens of such a composition may be present in a high percentage of a population exposed to the antigens, for example about 40% or more. In such an embodiment the average binding affinity and/or the average quantity of the antibodies may be in the upper tertile of binding affinity and/or quantity of antibodies produced by a patient. Known relative reactivities can be avidity or strength of reactivity with an antibody and/or activity state of a disease. A disease parameter may be exposure state (e.g., current or previous exposure) to a pathogen and/or an HSV (such as HSV-2), state of the infection (e.g., acute, latent, or recurrent) with a pathogen and/or an HSV (such as HSV-2), and immunity state (e.g., none, partial, or complete).

Another antigen composition of the inventive concept may include two or more antigens that are associated with a carrier, where at least two of the antigens have quantified and known relative reactivities with antibodies from sera of individuals affected by HSV-1 and HSV-2, and where at least two of the antigens have an association with a disease parameter. Such antigens may be selected from US3, US6, US8, US9, UL7, UL20, UL22, UL36 and UL44, and/or fragments thereof for HSV-1 and UL1, UL3, UL5, UL6, UL7, UL10, UL14, UL17, UL18, UL23, UL26, UL26.5, UL27, UL28, UL32, UL34, UL41, UL42, UL44, UL45, UL49, UL50, UL51, UL54, US6, US7, US8, US9 and US11, and/or fragments thereof for HSV-2. In some embodiments of the inventive concept antibodies to least two of the antigens of such a composition may be present in a high percentage of a population exposed to the antigens, for example about 40% or more. In such an embodiment the average binding affinity and/or the average quantity of the antibodies may be in the upper tertile of binding affinity and/or quantity of antibodies produced by a patient. Known relative reactivities can be avidity or strength of reactivity with an antibody and/or activity state of a disease. A disease parameter may be exposure state (e.g., current or previous exposure) to a pathogen and/or an HSV, state of the infection (e.g., acute, latent, or recurrent) with a pathogen and/or an HSV, and immunity state (e.g., none, partial, or complete).

In some embodiments of the inventive concept the carrier may be a pharmaceutically acceptable carrier suitable for use in a vaccine. In such an embodiment the vaccine may include four or more antigens and/or fragments thereof; such antigens or fragments thereof may be recombinant and/or at least partially purified. In another embodiment of the inventive concept the carrier may be a solid and/or insoluble phase suitable for use in a diagnostic assay, such as, for example, an array or microarray, antigens and/or fragments thereof may be disposed upon such a carrier. In such an embodiment the antigens or fragments thereof can be recombinant, and may be at a purity of at least 60% or least partially purified.

Various objects, features, aspects and advantages of the present invention will become more apparent from the following detailed description of preferred embodiments of the invention.

### BRIEF DESCRIPTION OF THE DRAWINGS

FIGS. 1A and 1B depict the expressible ORFs derived from an HSV genome (or ORFeome) by PCR and recombination cloning. FIG. 1A shows a gel of a PCR amplicon library, arranged by expected size. FIG. 1B shows a gel of corresponding DNA minipreparations after recombination cloning of PCR amplicons into pXi. C-pXi=control (non-recombinant) pXi plasmid.

FIG. 2 depicts a heat map of the HSV-1 and HSV-2 antibody profiles of human sera from infected and non-infected individuals, along with commercially supplied serum control materials. Columns correspond to sera used the probe an array of HSV and control antigens, with sample sizes shown at the bottom of the map. Serotype of the sera are shown at the top of the figure and used as the reference for sample categorization. The patient sera are classified in the map into seronegative, HSV-1 seropositive only, HSV-2 seropositive only, and HSV-1 and -2 seropositive groups. Rows in the heat map correspond to the antigens on the array, and are segregated into HSV-1 and HSV-2 antigens—listed to the right, ranked by descending average signal of the corresponding seropositive population.

FIGS. 3A and 3B depict comparisons results from HSV-1 or HSV-2 seropositive donors with those of seronegative donors. Histograms show array signals of seronegative, HSV-1 seropositive and HSV-2 seropositive donors. The responses to each antigen by HSV-1 seropositive donors (in FIG. 3A) and HSV-2 seropositive donors (in FIG. 3B) were compared to responses from seronegative donors by T tests, and the Benjamini-Hochberg corrected p values (pBH) shown overlaid onto the histograms.

FIG. 4 depicts comparisons between HSV-1 and HSV-2 seropositive donors. The responses to each antigen by HSV-1 seropositive donors are compared with those of HSV-2 seropositive donors.

FIGS. 5A and 5B depict a frequency of recognition (FR) analysis. Numbers of seropositives for each seroreactive antigen are shown as a percentage of the population. FIG. 5A shows seroreactive HSV-1 antigens recognized by >40% of HSV-1 seropositive population; antigens are ranked by descending frequency of the HSV-1 seropositive population. Calculated results of multiplexing US8/gE with UL22/gH and/or UL44/gC are shown in an inset to the right. FIG. 5B shoes seroreactive HSV-2 antigens recognized by >50% of HSV-2 seropositive population; antigens are ranked by descending frequency of the HSV-2 seropositive population. The strongest discriminatory antigens are indicated in both panels by an asterisk (*).

### DETAILED DESCRIPTION

The present inventive subject matter provides for the identification, analysis, and monitoring of antibody reactivity to specific HSV antigens, or antigen sets, which has diagnostic, prognostic, and therapeutic value, specifically with respect to various diseases and disorders. The present inventive subject matter also provides tools and methods to accurately survey HSV-induced infections, disorders, and diseases via the combination of: antibody reactivity detection and monitoring, and characterized sera samples.

It should be noted that in the following description antigens may be identified by either the gene descriptor for the gene that encodes the protein antigen and/or the name for the protein antigen. Thus, it should be understood that where the context indicates that a sequence or antigen is a protein sequence, a gene name for that sequence or antigen denotes the protein product for that gene. In some embodiments of the inventive concept, the HSV antigens have a sequence according to NCBI accession numbers NC 001806 and NC 001798 for HSV-1 strain 17 and HSV-2 strain 333, respectively. Where reference is made to antibodies it is recognized that while such antibodies may be referred to as having been obtained from serum, said antibodies may also derived from other sources, including, but not limited to, mucus, saliva, semen, lacrimal fluid, urine, feces, aqueous humor, pus, cerebrospinal fluid, lymphatic fluid, and synovial fluid.

The present inventive subject matter is also directed to the identification of specific HSV antigens that trigger antibody reactivity associated with various HSV diseases and disorders wherein the specific antigens have predetermined antibody reactivities from serum of a population of patients with a HSV disease or disorder. Thus, such specific antigens may have a statistically high probability to elicit antibody responses in a relatively large group of HSV-affected hosts.

In one aspect, the present inventive subject matter concerns a method of predicting the likelihood of a host being infected with (or having been exposed to) HSV-1, HSV-2, or both HSV-1 and HSV-2, by, at least in part, determining antibody reactivity against one or more antigens, or their variants, in a serum sample obtained from a host. In such an embodiment an antigen may be selected from the group consisting of: UL1, UL3, UL6, UL7, UL10, UL14, UL16, UL20, UL21, UL22, UL24, UL26, UL26.5, UL30, UL36, UL38, UL39, UL42, UL44, UL46, UL49, UL49.5, UL50, UL51, US1.5, US2, US3, US4, US6, US8, US8.5, US9, US10 and US11 from HSV-1, and UL1, UL3, UL5, UL6, UL7, UL10, UL11, UL14, UL16, UL17, UL18, UL23, UL26, UL26.5, UL27, UL28, UL32, UL34, UL36, UL41, UL42, UL44, UL45, UL46, UL49, UL50, UL51, UL54, US6, US7, US8, US8A, US9, US10, and US11 from HSV-2. The presence of one or more antibody(ies) reactive against one or more of UL1, UL3, UL6, UL7, UL10, UL14, UL16, UL20, UL21, UL22, UL24, UL26, UL26.5, UL30, UL36, UL38, UL39, UL42, UL44, UL46, UL49, UL49.5, UL50, UL51, US1.5, US2, US3, US4, US6, US8, US8.5, US9, US10 and US11 from HSV-1 and UL1, UL3, UL5, UL6, UL7, UL10, UL11, UL14, UL16, UL17, UL18, UL23, UL26, UL26.5, UL27, UL28, UL32, UL34, UL36, UL41, UL42, UL44, UL45, UL46, UL49, UL50, UL51, UL54, US6, US7, US8, US8A, US9, US10, and US11 from HSV-2 indicates an increased likelihood of the host being infected with and/or having been exposed to HSV.

In one aspect, the present inventive subject matter concerns a method of predicting the likelihood of a host being infected by HSV-1 and not HSV-2, comprising determining antibody reactivity against one or more antigens, or their variants, from a serum sample obtained from a host, wherein the antigen is selected from the group consisting of US3, US6, US8, US9, UL7, UL20, UL22, UL36 and UL44 from HSV-1.

In another aspect, the present inventive subject matter concerns a method of predicting the likelihood of a host being infected by HSV-2 and not HSV-1, comprising determining antibody reactivity against one or more antigens, or their variants, in a serum sample obtained from a host, wherein the antigen is selected from the group consisting of UL1, UL3, UL5, UL6, UL7, UL10, UL14, UL17, UL18, UL23, UL26, UL26.5, UL27, UL28, UL32, UL34, UL41, UL42, UL44, UL45, UL49, UL50, UL51, UL54, US6, US7, US8 US9 and US11 from HSV-2.

In one aspect, the present inventive subject matter concerns a method of predicting the likelihood of a patient having a HSV-induced infection, disease, or disorder, comprising determining antibody reactivity against one or more HSV antigens, or their variants, in a serum sample obtained from a host, wherein the antigen is selected from a specific set of antigens; wherein antibody reactivity against one or more of said specific antigens indicates an increased likelihood of the host having a HSV-induced disease or disorder.

In various embodiments, the reactivity level of at least 2, or at least 5, or at least 10, or at least 15, or at least 20, or at least 25 antibodies is determined. Reactivity of antibodies with the antigens of the inventive concept may be determined by characterization of radiation, transmitted or reflected light, enzymatic activity, fluorescence, light scatter, fluorescence polarization, phosphorescence, chemiluminescence, electrochemiluminescence, and/or amperommetric signals by appropriate instrumentation. For example, an ELISA may be performed in a microwell plate and the results measured colorimetrically in a commercial microplate reader. In another example, a test strip result may be read by visual inspection or may be inserted into a scanner.

While determination of reactivity can be performed in numerous formats well known in the art, it is generally preferred that determination is done in a multiplex format, and especially in array, ELISA, or strip assay or “dipstick” formats. An array may be essentially planar, with individual antigens fixed as discrete “spots” on a test surface. Antibody reactivity on such a planar array can be, for example, characterized using a scanner or CCD camera that detects color changes at the test spots. Alternatively, the multiplex format may comprise a fluid array, in which antibody reactivity levels are determined for a discrete population of suspended microparticles that are coupled with antigens. Antibody reactivities may be characterized with such a fluid array using a flow detector, such as, for example, a cell sorter that characterizes fluorescence emitted from suspended microparticles as they move past a detector. Thus, arrays or strip assays having at least one, more typically at least two, even more typically at least 5, or at least 10, or at least 15, or at least 20, or at least 25 antigens are contemplated. ELISAs, or strip assays having at least one, more typically at least three are also contemplated.

In another aspect, the present inventive subject matter concerns a method of predicting the likelihood of a host having an HSV infection, disease, or disorder, comprising determining prognostic antibody reactivity against one or more specific HSV antigens, or their variants, in a serum sample obtained from the host, and normalized against the level(s) of one or more non-prognostic antibody reactivity(ies) in the serum sample that act as an internal control(s). Alternatively, a reference reagent set may utilized that provides one or more external control levels of antibody reactivity that may be used for normalization. In such embodiments antibody reactivity against one or more of said specific HSV antigens indicates an increased likelihood of the host having a disease or disorder.

In another aspect, the present inventive subject matter concerns a method of predicting the likelihood of a host having an HSV infection, disease, or disorder, comprising determining prognostic antibody reactivity against one or more HSV antigens presented herein above, or their variants, in a serum sample obtained from the host, and normalized against the level(s) of one or more non-prognostic antibody reactivity(ies) in the serum sample that act as an internal control(s). Alternatively, a reference reagent set may utilized that provides one or more external control levels of antibody reactivity that may be used for normalization. In such embodiments antibody reactivity against one or more of said specific HSV antigens indicates an increased likelihood of the host having a disease or disorder.

In a different aspect, the inventive subject matter comprises two or more of the HSV antigens presented herein above, immobilized on a surface, wherein the HSV antigens may be associated with a single disease or more than one disease. In some embodiments of the inventive concept a test surface may comprise HSV antigen variants, including fusion peptides, truncated forms, conjugated forms, non-glycosylated forms, multimeric forms, recombinant forms, chimeric forms, etc.

In one embodiment, the inventive subject matter concerns a method of predicting the likelihood of a patient being infected by HSV by:

(a) determining the reactivity levels of antibodies against HSV antigens, or their variants, presented herein above, in a sample obtained from the patient, optionally normalizing the determined reactivity levels against the reactivity levels of other antibodies against HSV antigens (or their variants), in said sample, or against a reference set of antibody reactivity levels;

(b) subjecting the data obtained in step (a) to statistical analysis; and;

(c) determining the likelihood of said patient being infected by HSV.

In a still further aspect, the inventive subject matter concerns a method of preparing a personalized proteomics and antibody profile for a HSV patient by:

(a) subjecting a serum or other sample obtained from the patient to protein array analysis;

(b) determining the reactivity level of one or more antibodies in the sample against HSV antigens, or their variants, wherein the reactivity level is optionally normalized against control reactivity levels; and

(c) creating a report summarizing the data obtained by the analysis.

The report may include prediction of the likelihood of the severity of and/or probable stage of HSV infection in the patient. In some embodiments of the inventive concept the report can include recommendation of a treatment modality for said patient.

In a further aspect, the inventive subject matter concerns a method for detecting and/or characterizing one or more HSV antibodies in a patient. The present inventive subject matter also provides tools and methods to accurately survey HSV infections via the combination of: antibody detection and monitoring, and characterized sera samples. Such surveys may, for example, provide information related to the progress of HSV disease in an individual, which may be valuable to a clinician in selecting appropriate treatment modes. Alternatively, when applied to a population such surveys may provide information related to the epidemiology of HSV strains within a population, thereby providing valuable information useful to diagnosis and control of the spread of HSV infection.

Detection and/or characterization of HSV antibodies in a patient sample may be facilitated by associating the HSV antigens comprising the detection reaction with a carrier. In such embodiments of the inventive concept, the carrier may be a solid or insoluble (i.e. particulate) carrier, and the plurality of HSV antigens is disposed on the carrier in an array. It is further contemplated that the antigens or fragments thereof may be in crude expression extracts, in partially purified form (e.g., purity of less than 60%), or in highly purified form (purity of at least 95%). In some embodiments of the inventive concept such HSV antigens, or fragments thereof, may be purified to about 1%, 2%, 5%, 10%, 20%, 30% 40%, 50%, 60%, 70%, 80%, 90%, 95%, or 99% or greater purity. The antigens in such arrays may be recombinant or native. Alternatively, solid phases need not be limited to planar arrays, but may also include microwell plates, beads, microparticles, magnetically responsive particles, columns, dipstick-type formats, microfluidic devices, etc.

Antigens identified herein have, advantageously, been identified as both immunogenic and capable of generating a protective immune response in humans by virtue of identification using antibodies from infected individuals. Furthermore, such antigens have not undergone eukaryotic processing, such as glycosylation, and are therefore amenable to large scale production. As such, the antigens disclosed herein may have utility as therapeutic and/or protective vaccines. In another aspect of the inventive subject matter, therefor, HSV antigens that are identified as triggering significant antibody reactivity may be utilized in an antigen composition. In such an embodiment the antigen composition can comprise two or more reactive antigens of a HSV-induced infection, disease, or disorder and are associated with a carrier, where such antigens have quantified and known relative reactivities with respect to sera of a population infected with HSV, and wherein the antigens have a known association with a HSV disease parameter. In a preferred embodiment the antigens are polypeptides or fragments thereof.

Such vaccine embodiments of the inventive concept can be facilitated by associating one or more antigens with a carrier. In such embodiments of the inventive concept, therefore, the carrier can be a pharmaceutically acceptable carrier, and such a composition may be formulated as a vaccine. In such an embodiment it is generally preferred that the vaccine comprises multiple antigens. In such embodiments a vaccine may include, 2, 3, 4, 5, 6, 7, 8 9, 10, or more antigens. In a preferred embodiment of the inventive concept the vaccine comprises four antigens. Alternatively, a vaccine of the inventive concept may include a single antigen. Depending on the particular HSV-induced disease or disorder, it is contemplated that the HSV antigens, or fragments thereof, are at least partially purified and/or recombinant. Such HSV antigens, or fragments thereof, may be purified to about 10%, 20%, 30% 40%, 50%, 60%, 70%, 80%, 90%, 95%, or 99% or greater purity.

### Examples

The inventors have discovered numerous HSV antigens that were capable of triggering antibody reactivity from a variety of types and stages of HSV infection, utilizing microarrays to perform proteome screening of serum samples from infected populations. Antigens according to the inventive subject matter were presented herein, and it is contemplated that such antigens can be used by themselves, or more preferably, in combination with other antigens in the manufacture of a diagnostic devices, therapeutic compositions, and vaccines.

Exemplary Methods

Serum Samples. Blood samples were collected from healthy donors and from patients attending the UCI Medical Center for diagnosis and treatment of herpes infections. All infections were latent and no acute cases were examined. All samples were collected as part of an immunological study with consent and under local IRB approval. For the present study, the serum fraction was separated from each blood sample and stored at −80° C. prior to use. All sera were assayed by FDA-approved commercial HSV-1 and HSV-2 ELISAs (HerpeSelect®; Focus Diagnostics) according to the manufacturer's instructions. Control sera representing the general adult population in the same geographical location as the patients (Orange County, CA) were collected by the UCI General Clinical Research Center (GCRC) with consent and under local IRB approval.

Construction of HSV-1 and HSV-2 Proteome Microarrays. Proteome microarrays were fabricated by PCR amplification of coding sequences in genomic DNA and insertion of amplicons into a T7 expression vector by homologous recombination, followed by expression in coupled transcription-translations in vitro (IVTT) and direct printing onto microarrays (Davies, D. H., et al. 2005. Proc Natl Acad Sci USA 102:547-52; Luevano, M., et al. 2010. Virology 405:31-40). Gene sequences for PCR primer design were obtained from NCBI (Accession numbers NC_001806 and NC_001798 for HSV-1 strain 17 and HSV-2 strain 333, respectively). Gene nomenclature used is as published in the curated Oral Pathogen Genome Sequence Databases (ORALGEN) at the Los Alamos National Laboratory (http://www.oralgen.lanl.gov/). Template DNA was a generous gift from Dale Carpenter and Steve Wechsler, University of California, Irvine Department of Ophthalmology. HSV-1 strain 17 DNA was supplied as 5 overlapping genomic fragments cloned into cosmids. HSV-2, strain 333 DNA was prepared from virion-extracted DNA or purchased from ATCC (Manassa, Va.).

Primers used for PCR amplification contained 20 bp nucleotide specific for each gene with an extension of 20 bp complementary to ends of linear pXT7 vector at 5 prime ends. For PCR, genes were amplified using AccuPrime™ GC-Rich DNA polymerase (Invitrogen, Grand Island, N.Y.) or Phusion® High-Fidelity PCR Master Mix with GC Buffer (Thermo Scientific, Waltham, Mass.) with an addition of DMSO (final concentration 2%) and 8 ng/μl BSA, using touchdown PCR with cycling conditions of initial denaturation at 98° C./1 min, followed by 20 cycles of 98° C./10 sec, 68° C./20 sec with decremental temperature of 0.5° C./cycle, and 72° C. for 30 sec/kb, followed by 20 cycles of 98° C./10 sec, 58° C./20 sec and 72° C./30 sec/kb. In vivo homologous recombination takes place between the PCR product and pXT7 vector in competent DH5a cells. The recombinant plasmids were isolated from this culture using QIAprep 96 Turbo kit (Qiagen, Venlo, Netherlands). All recombinant plasmids were confirmed as containing insert by QC-PCR, in which a band of expected size was amplified from the recombinant using the same primers as used in the original PCR. Plasmids that generated strong signal on the array were also confirmed by sequencing.

For array fabrication, purified minipreparations of DNA were expressed using an E. coli based in vitro transcription/translation expression system (Invitrogen Expressway™ Cell-Free Expression System, Invitrogen, Grand Island, N.Y.). Twenty μL reactions were prepared in sealed 384 well microwell plates and incubated for 16 hours on a platform shaker at 250 rpm at 24° C. A protease inhibitor cocktail (Roche Complete, Roche, Penzberg, Germany) and Tween-20 to a final concentration of 0.05% were added prior to printing. The expressed protein reactions were printed in singlicate without further purification onto 8-pad nitrocellulose-coated Oncyte Nova slides (Grace Bio-Labs, Bend, Oreg.) using an OmniGrid Accent 100 microarray printer (Genomic Solutions, Ann Arbor, Mich.) in 1×4 sub-array format. Each sub-array included multiple negative control spots comprising mock expression reactions that lacked a DNA template. Each sub-array also included positive control spots of four serial dilutions of a mixture of mouse, rat and human IgG and two serial dilutions of human IgM. These positive and negative controls were used to normalize the data from different arrays. Arrays also included four serial dilutions of purified recombinant Epstein-Barr virus nuclear antigen-1 (EBNA-1; DevaTal Inc., Hamilton, N.J.) as a guide to serum quality.

Protein expression in each spot was monitored using antibodies against the N-terminal poly-His (clone His-1, Sigma-Aldrich, St. Louis, Mo.) and the C-terminal HA (clone 3F10, Roche, Penzberg, Germany) tags engineered into each protein. Arrays were first blocked for 30 minutes in Protein Array Blocking Buffer (Whatman, Little Chalfont, United Kingdom) at ambient temperature and then probed for 1 hour with anti-tag antibodies diluted 1/1,000 in the blocking buffer. The slides were then washed 3× in tris(hydroxymethyl)aminomethane (Tris)-buffered saline containing 0.05% (v/v) Tween 20, (T-TBS) and incubated with biotinylated secondary antibodies (Jackson ImmunoResearch, West Grove, Pa.). After washing the slides 3× in T-TBS, bound antibodies were detected by incubation with streptavidin-conjugated SureLight® P-3 (Columbia Biosciences, Columbia, Md.). Slides were then washed 3× in T-TBS followed by 3× in TBS, and dipped in distilled water prior to air drying by brief centrifugation. Slides were scanned in a Perkin Elmer ScanArray confocal laser scanner and data acquired using ScanArrayExpress software (Waltham, Mass.).

Since the protein concentrations of each spot are not controlled in this type of array, different proteins may have varying concentrations and caution must be exercised when comparing the signal intensities of different proteins by a given sera sample. However, since all the arrays are printed together, the concentration of a given protein is constant between different arrays. Thus, comparison of signal intensities of a given protein between different sera can be made, and are directly correlated with antibody titer.

For probing with human sera, samples were diluted to 1/100 in 1× Protein Array Blocking Buffer supplemented with E. coli lysate (Antigen Discovery, Inc., Irvine, Calif.) at a final concentration of 10 mg/ml protein to block anti-E. coli antibodies, and incubated at 37° C. for 30 minutes with constant mixing. Meanwhile the arrays were blocked with Protein Array Blocking Buffer for 30 minutes. The blocking buffer was removed and the arrays were probed with the pretreated sera overnight at 4° C. with gentle rocking. The slides were then washed 3× in T-TBS and incubated in biotinylated anti-human IgG Fc (Jackson Immuno Research, West Grove, Pa.) diluted 1/200 in Protein Array Blocking Buffer. After washing the slides 3× each in T-TBS and TBS, bound antibodies were visualized as described above. Control arrays were similarly probed except that the primary antibody was replaced by Protein Array Blocking Buffer supplemented with E. coli lysate.

Array Data Analysis and Statistical Treatment. Raw data were collected as the mean pixel signal intensity data for each spot. Negative and positive control spots (the ‘no DNA’ and IgG spots, respectively) were used to perform variance stabilization normalization (VSN) using the “VSN” package in the statistical environment R from the Bioconductor suite (http://Bioconductor.org/). Reactive antigens were defined as positive when the normalized signal intensity was above the mean of the ‘no DNA’ control spots plus two standard deviations (C+2SD). P-values were calculated for the log normalized data by comparing groups of donors using a Bayes-regularized t-test. To account for multiple test conditions p-value adjustments were calculated by the Benjamini-Hochberg method (Benjamini, Y., and Y. Hochberg. 1995. J. Roy. Stat. Soc., B 57:289-300) to give BH p-values. Positive antigens were classified as type-specific or cross-reactive according to significance (BH p-value<0.05 and >0.05, respectively). Receiver Operator Characteristic (ROC) analyses were performed with log transformed/normalized array data for single antigens by testing signals for each donor as threshold cut off to discriminate HSV-1 and HSV-2 infection. To calculate the area under the curve (AUC) for each antigen, receiver Operator Characteristic (ROC) analyses were performed with normalized array data to discriminate HSV-1 and HSV-2 infection. The area under the curve (AUC) was used as a relative measure of each antigen's ability to discriminate between HSV-1 and -2 infections. For frequency of recognition (FR) analysis, a cutoff was defined for each antigen on the array using the normalized data, and set as the average signal+3SD of the seronegative population as defined by HerpeSelect ELISA (Focus Diagnostics, Cypress, Calif.). The numbers of individuals above the cutoff in each of the seropositive groups were determined and expressed as a percentage.

Enrichment analysis. Each protein encoded by HSV-1 (strain 17) was assigned one or more gene ontology (GO) component in accordance with the curated database at www.uniprot.org. The numbers of ORFs in the genome and in the seroreactive antigens belonging to each GO classification were defined as percentages, and the ratio of genome vs. seroreactive used to determine fold-enrichment. GO classifications that were enriched by 1.5-fold or more were considered enriched among the seroreactive antigens relative to the proteome. P values for enrichment statistical analysis were calculated using Fisher's exact test in the R environment.

Exemplary Results

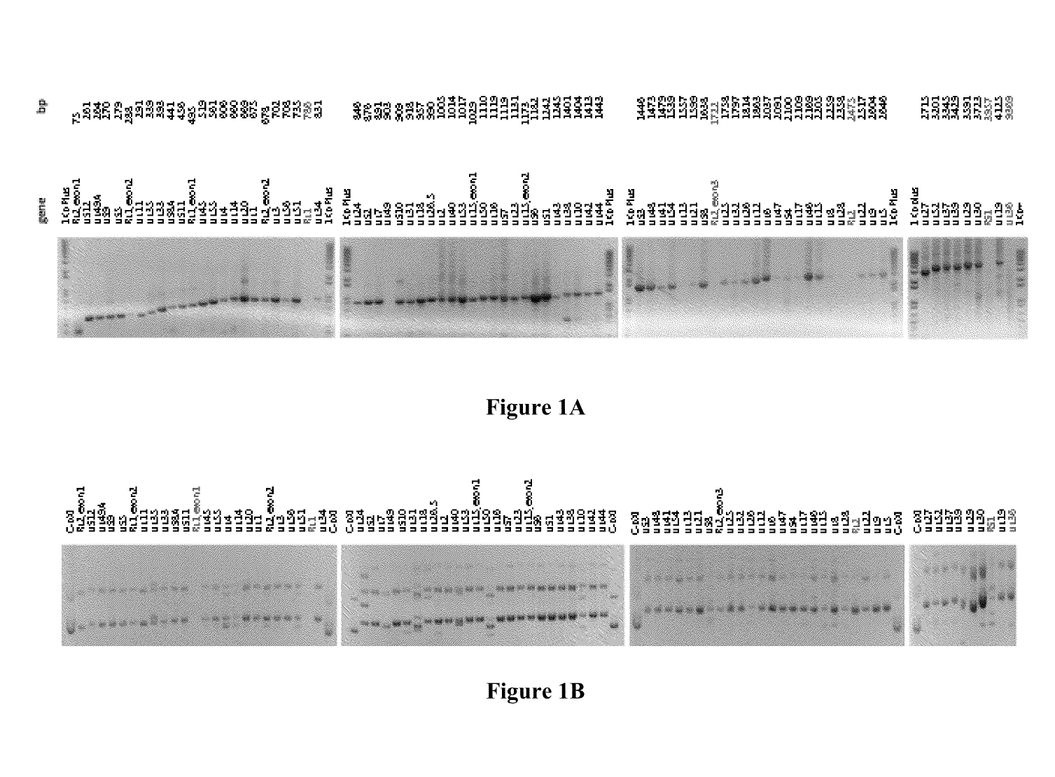

Exemplary results from construction of a library of expressible HSV open reading frames (an “ORFeome”) are depicted in FIGS. 1A and 1B. FIG. 1A shows results from gel electrophoresis of a PCR library arranged by expected size. Some genes failed to amplify under any of the PCR conditions used, as shown by the negative results in the corresponding gel lane. FIG. 1B shows results of gel electrophoresis of corresponding HSV-2 and representative of HSV-1 DNA minipreparations after recombination cloning of PCR amplicons into pXi. All plasmids are circular/non-linearized. In this figure, C-pXi=control (non-recombinant) pXi plasmid. Genes that failed to clone generally correspond to those genes that could not be amplified.

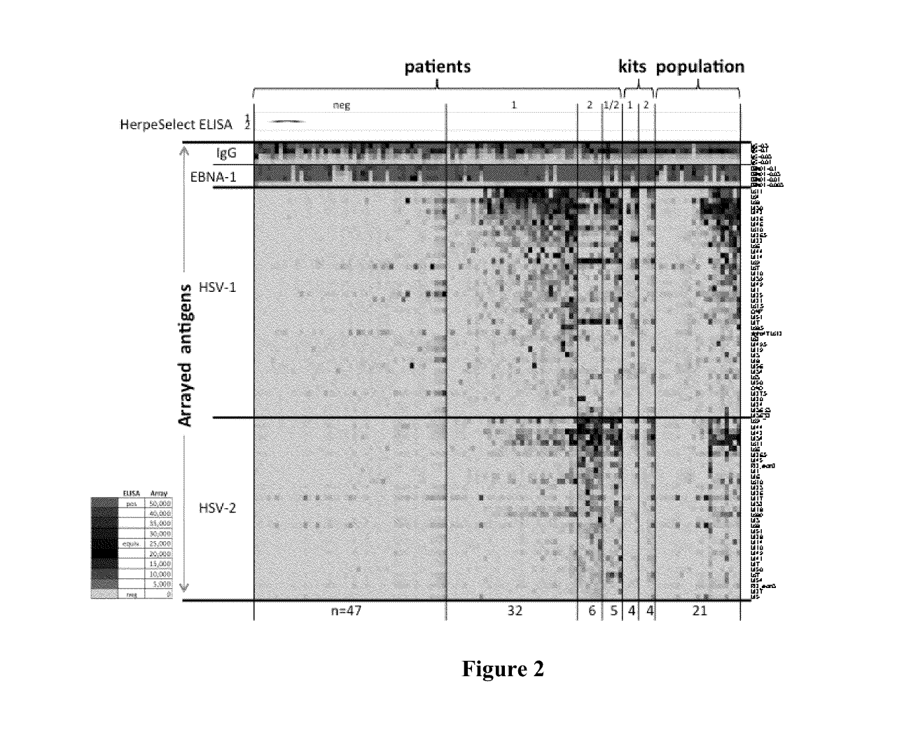

A heat map overview of HSV-1 and HSV-2 antibody profiles from human serum samples reacted with an HSV-1 and HSV-2 antigen array is shown in FIG. 2. Columns correspond to individual serum samples used to the probe the array, with sample sizes shown at the bottom of the map. Results of serotyping using HerpeSelect-1 and -2 IgG ELISAs (Focus Diagnostics, Cypress, Calif.) are also shown in the indicated rows of the map, and were used as the reference for sample categorization. The patient sera were classified as seronegative (‘neg’ n=47), HSV-1 seropositive only (‘1’ n=32), HSV-2 seropositive only (‘2’ n=6), and HSV-1 and -2 seropostive (1/2, n=5) groups. Control sera supplied with each ELISA kit (‘kits’, n=4 each), comprising negative, equivocal and two positive samples, were also probed on arrays. For comparison, sera from the general population were probed (‘population’, n=21). With the exception of the ELISA results, rows in the heat map correspond to the antigens on the array. These were segregated into HSV-1 and HSV-2 antigens, and are listed on the right side of the map. Only data from antigens that were reactive against sera from the HSV-1 or HSV-2 seropositive populations are shown. An antigen was defined as reactive when the average signal intensity for either population was greater than the average+2SD value of the control spots consisting of in vitro translation reactions that lacked DNA template, as described above (C+2SD). Other antigens shown are: (1) purified, titrated human IgG, which is recognized by the secondary antibody and is used to help monitor inter-array probing, and (2) purified, titrated Epstein Barr Nuclear Antigen-1 (EBNA-1) which serves as a control for serum quality as most individuals are seropositive for this antigen. The heat map was generated from raw array data from which the signal of the of background control spots was subtracted, and the antigens ranked by descending average signal of the corresponding seropositive population. The sera are also ranked within each of the 7 groups by increasing average signal.

ELISA seronegative clinical samples show only slight reactivity against either HSV-1 or HSV-2 antigens on the array. In contrast, most of the HSV-1 seropositive donors showed elevated reactivity to HSV-1 antigens relative to the seronegative donors, and minimal cross-reactivity with HSV-2 antigens. The breadth of the profile of the HSV-1 seropositive group was highly variable, which may correlate with clinical symptoms. In contrast to the HSV-1 seropositive group, which shows very specific reactivity for the HSV-1 antigens, the HSV-2 seropositive donors showed elevated reactivity to both HSV-2 and HSV-1 antigens, consistent with cross-reactivity for HSV-1. As expected, reactivity by the HSV-1/HSV-2 double positive donors was equally distributed between the HSV-1 and HSV-2 antigens. Finally, about half of the general population (represented by the 21 samples) appeared to be seropositive for HSV-1. Seroprevalence for HSV-2 in the general population was slightly lower, and two individuals showed particularly broad profiles. The concordance of the array data compared to the reference ELISA serotyping is presented below in the frequency of recognition (FR) analysis.

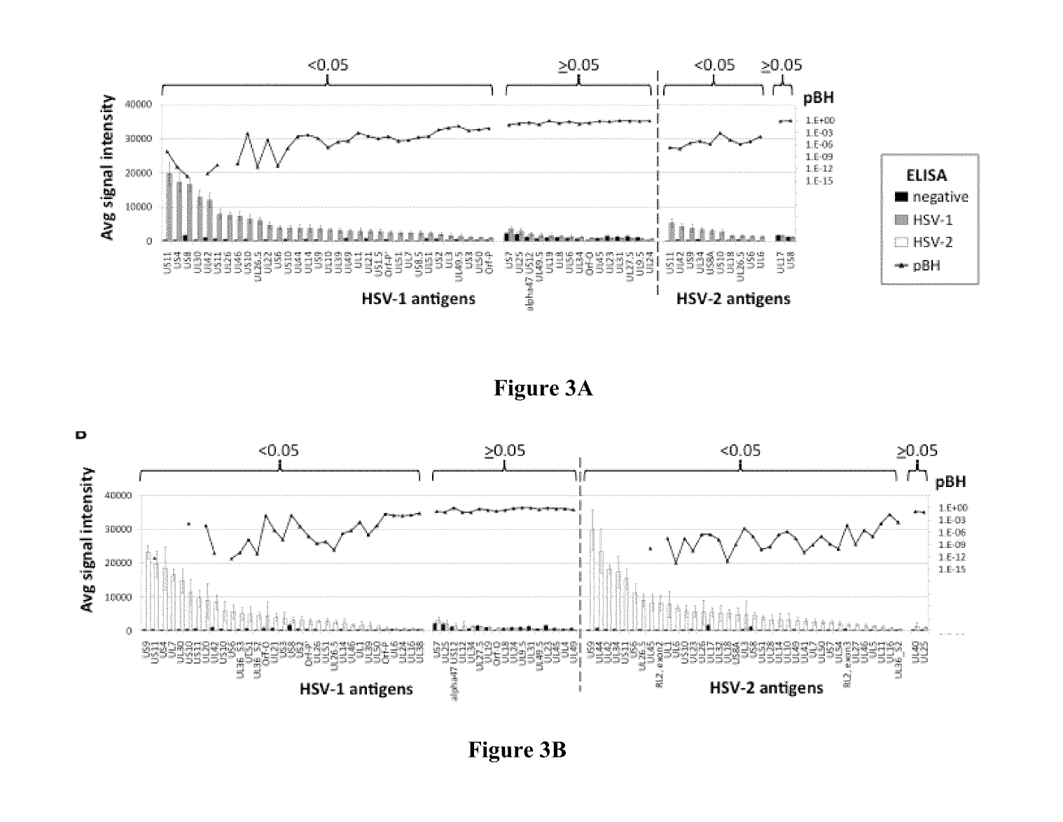

FIGS. 3A and 3B depict comparisons between the antibody reactivity profiles of HSV-1 or HSV-2 seropositive donors and the antibody reactivity profile of seronegative donors. Histograms show average array signals+SEM of seronegative (black fill), HSV-1 seropositive (gray fill), and HSV-2 seropositive (no fill) donors. Only the seroreactive antigens (i.e., those for which average signal exceeds C+2SD) are shown. FIG. 3A shows a comparison of results from HSV-1 seropositive donors, and FIG. 3B shows a comparison of results from HSV-2 seropositive donors to those of seronegative donors using T tests. Benjamini-Hochberg corrected p values (pBH) are shown overlaid onto the histograms and were used to classify the antigens into significant and non-significant responses (pBH<0.05 and >0.05, respectively). Gaps in the pBH line indicate corrected p values<10-30.

This comparison between HSV-1 seropositive and the seronegative donors reveals several significant HSV-1 antigens. Non-significant antigens (i.e., those that gave similar signals in seronegatives and seropositives) were all low signals and close to the cutoff (C+2SD), and could be eliminated using more stringent criteria. A number of HSV-2 antigens were also recognized by the HSV-1 seropositive donors, although the signals were relatively low, providing further confirmation that sera from the HSV-1 only population shows only modest cross-reactivity with HSV-2 antigens. Comparison between the HSV-2 seropositive and seronegative donors (FIG. 3B) also reveals several significant HSV-2 antigens. However, sera from HSV-2 donors also showed extensive cross-reactivity with HSV-1 antigens. To determine whether any of these significant antigens had potential diagnostic utility for discriminating between HSV-1 and HSV-2 infections it was found to be necessary to compare the HSV-1 antibody profiles with HSV-2 antibody profiles.

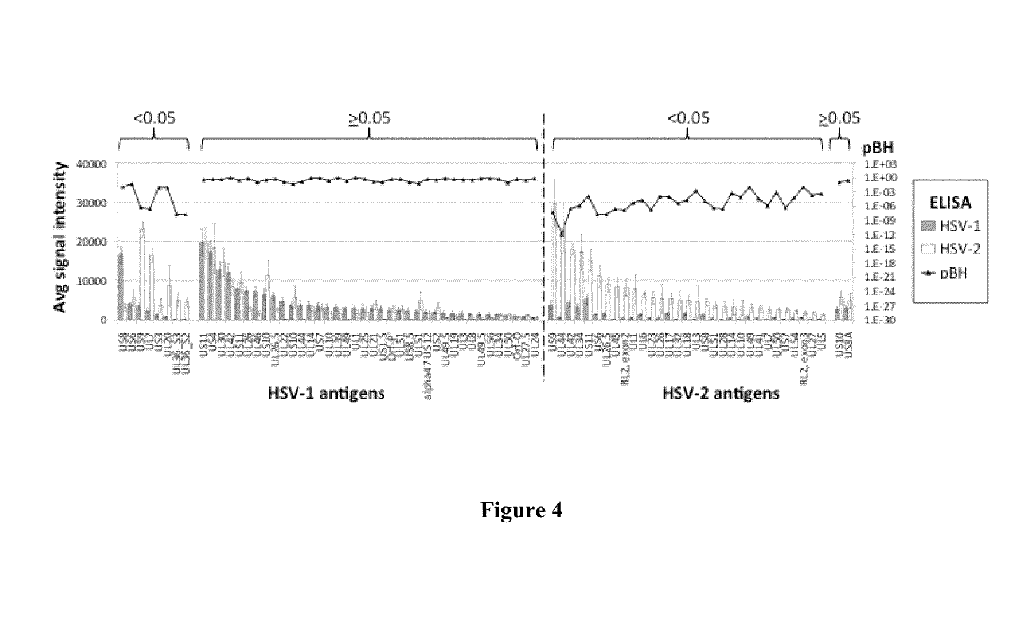

FIG. 4 depicts comparisons between antibody reactivity profiles of HSV-1 seropositive donors and HSV-2 seropositive donors. As in FIG. 3, T-tests were performed and Benjamini-Hochberg corrected p values (pBH) are shown overlaid onto the histograms. Surprisingly, the majority of the reactive HSV-1 antigens were not significant, confirming the observation made from the heat map that HSV-1 antigens were recognized by sera from both HSV-1 and HSV-2 seropositive donors. Seroreactive HSV-1 antigens are listed and classified by T-tests of comparative results between HSV-1 and HSV-2 seropositive donors into significant or discriminatory (pBH<0.05) and non-significant or cross-reactive (pBH>0.05) in Table 1, in the same order as they are shown in FIG. 4. Protein identities were obtained from Uniprot (http://www.uniprot.org/) and Oralgen (http://www.oralgen.lanl.gov/) with additional annotation from current mass spectrometry data.

TABLE 1

| | ORF | Protein description |
|---|---|---|
| pBH < 0.05 | US8 | glycoprotein E, gE |
| | US6 | glycoprotein D, gD |
| | US9 | envelope protein |
| | UL7 | virion protein |
| | US3 | serine/threonine protein kinase cellular homolog, KR1 |
| | UL20 | membrane protein |
| | UL36 | very large tegument protein, ICP1/2 (335,890 Da) |
| pBH ≧ 0.05 | US11 | RNA-binding tegument protein, DNB |
| | US4 | glycoprotein G, gG |
| | UL30 | DNA-directed polymerase cellular homolog, DPOL |
| | UL42 | DNA polymerase accessory protein, VPAP |
| | UL26 | serine protease, VP40, self-cleaves to form VP21 and VP24 |
| | UL46 | tegument protein, VP11/12; modulates alphaTIF |
| | US10 | tegument protein |
| | UL26.5 | capsid scaffolding protein, ICP35, VP22a |
| | UL22 | glycoprotein H, gH |
| | UL44 | glycoprotein C, gC |
| | UL14 | minor tegument protein |
| | US7 | glycoprotein I, gI |
| | UL10 | glycoprotein M, gM |
| | UL39 | ribonucleotide reductase large subunit cellular homolog, ICP6, RIR1 |
| | UL49 | putative microtubule-associated protein, MAP, VP22 |
| | UL1 | virion glycoprotein L, gL |
| | UL25 | DNA packaging virion protein |
| | UL21 | tegument protein |
| | US1.5 | |
| | Orf-P′ | |
| | UL51 | tegument protein |
| | US8.5 | |
| | US12 | TAP1/2 binding protein, ICP47, IE12 |
| | US2 | tegument protein |
| | UL49.5 | membrane-associated virion protein |
| | UL19 | major capsid protein, VP5, ICP5 |
| | UL3 | nuclear phosphoprotein cellular homolog |
| | UL8 | DNA helicase/primase complex associated protein (HEPA) |
| | UL56 | virulence-associated virion protein |
| | UL34 | membrane-associated virion protein |
| | UL50 | deoxyuridine triphosphatase, dUTPase (DUT) |
| | Orf-O | |
| | UL27.5 | |
| | UL24 | putative membrane-associated protein with syn-locus |

Note that while several of these antigens are identified as glycoproteins, the polypeptides that formed the basis of these results were not glycosylated. Many of these antigens also represent proteins that are not expressed on the surface of HSV-1, and would not normally be considered candidates for assay or vaccine development by conventional methodologies.

As can be seen in Table 1, few HSV-1 antigens were significant, and the signal intensity for only one of these (US8/glycoprotein E) was observed to be greater in HSV-1 seropositive donors. The remaining significant antigens gave greater signal intensities with sera from HSV-2 seropositive donors. This unanticipated result may be due to the cross reactivity of sera from HSV-2 seropositive donors. For example, the HSV-2 US9 tegument protein is strongly recognized in HSV-2 infection, whereas the corresponding HSV-1 antigen is not strongly recognized in HSV-1 infection. UL7 shows similar behavior. The homologies between these HSV-1 and -2 orthologs are high, suggesting that antibodies are likely to cross-react.

Results with reactive HSV-2 antigens were found to differ from those of HSV-1, as the majority gave significantly higher signals with the HSV-2 seropositive donors. Seroreactive HSV-2 antigens are listed and classified by T-tests of comparative results between HSV-2 and HSV-1 seropositive donors into significant or discriminatory (pBH<0.05) and non-significant or cross-reactive (pBH>0.05) in Table 2, in the same order as they are shown in FIG. 4. Protein identities were obtained from Uniprot (http://www.uniprot.org/) and Oralgen (http://www.oralgen.lanl.gov/) with additional annotation from current mass spectrometry data.

| | ORF | Protein description |
|---|---|---|
| PBH < 0.05 | US9 | envelope protein |
| | UL44 | glycoprotein C, gC |
| | UL42 | DNA polymerase accessory protein |
| | UL34 | ?membrane-associated virion protein |
| | US11 | RNA-binding tegument protein (DNB) |
| | US6 | glycoprotein D, gD |
| | UL26.5 | capsid scaffolding protein, ICP35, VP22a |
| | UL45 | membrane protein type II |
| | RL2 | promiscuous transactivator, cellular homolog, ICP0, VMW110, IE110 (ICP0) |
| | UL1 | glycoprotein L, gL |
| | UL6 | virion protein |
| | UL23 | thymidine kinase, ICP36 |
| | UL26 | serine protease, self-cleaves to form VP21 and VP24 (VP40) |
| | UL17 | capsid protein |
| | UL32 | cleavage and packaging protein |
| | UL18 | capsid protein, VP23 |
| | UL3 | nuclear phosphoprotein cellular homolog |
| | US8 | glycoprotein E, gE |
| | UL51 | tegument protein |
| | UL28 | DNA cleavage and packaging protein, ICP18.5 |
| | UL14 | minor tegument protein |
| | UL10 | glycoprotein M, gM |
| | UL49 | putative microtubule-associated protein, MAP, VP22 |
| | UL41 | virion host shutoff protein, cellular homolog, VHS |
| | UL7 | virion protein |
| | UL50 | deoxyuridine triphosphatase, dUTPase |
| | US7 | glycoprotein I, gI |
| | UL54 | regulates and transports RNA, IE63, VMW63, ICP27, IE63 |
| | UL27 | glycoprotein B, gB, VP7 |
| | UL5 | component of DNA helicase-primase complex, cellular homolog. HELI |
| pBH > 0.05 | US8.5 | |
| | US10 | tegument protein |

Only two antigens (US8.5 and US10) were non-significant or cross-reactive with sera from HSV-1 seropositive donors. Note that while several of these antigens are identified as glycoproteins, the polypeptides that formed the basis of these results were not glycosylated. Many of these antigens also represent proteins that are not expressed on the surface of HSV-2, and would not normally be considered candidates for assay or vaccine development by conventional methodologies.

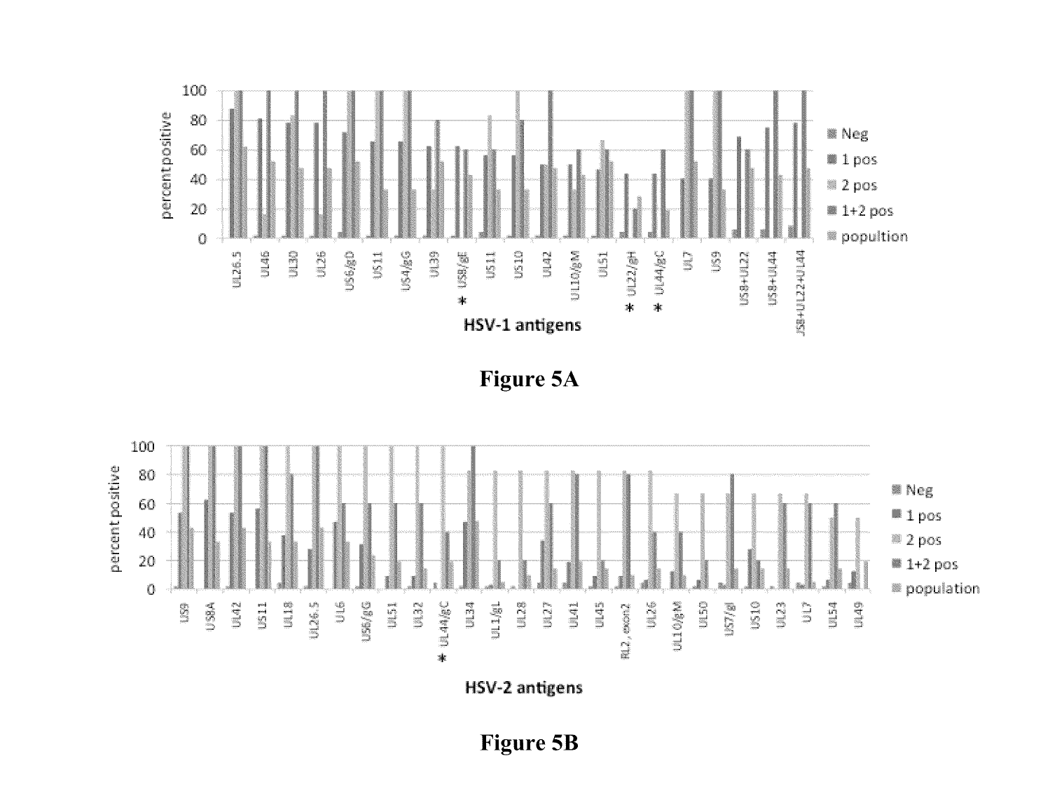

While the T tests provide a guide to the antigens that discriminate between HSV-1 and HSV-2 seropositive populations, for diagnostics it is important also to determine the frequency of recognition (FR) of these candidate discriminatory antigens. Typical results from such a frequency of recognition (FR) analysis are shown in FIGS. 5A and 5B. The cut-off value for recognition was defined as the mean+3 SD of the log transformed/normalized data for the seronegative population. The numbers of seropositives for each seroreactive antigen were then determined in each population and the value converted to a percentage of that population. The most discriminatory antigens are shown in both panels by an asterisk (*).

FIG. 5A shows seroreactive HSV-1 antigens recognized by more than 40% of the HSV-1 seropositive population. As shown, the antigens are ranked by descending frequency of recognition within the HSV-1 seropositive population. The majority of the antigens recognized were highly cross-reactive with the HSV-2 seropositive population. It is notable that such antigens may have utility for tests designed to diagnose HSV infection in general without discriminating between HSV-1 and HSV-2. For a HSV-1 specific test, only 3 reactive antigens (glycoproteins E, H and C) showed acceptably low cross-reactivity with the HSV-2 seropositive donors. Of these, HSV-1 US8/gE, which was determined earlier by T tests as the HSV-1 antigen best able to discriminate between HSV-1 and HSV-2 infection, had the best FR values of 62.5% and 0% in HSV-1 and HSV-2 seropositives, respectively. The two other identified antigens (UL22/gH and UL44/gC) had FR values of 43.8% and 0% in HSV-1 and HSV-2 seropositive donors, respectively. The highest FR was achieved by combining all three antigens (78.1% vs 0% of HSV-1 and HSV-2 seropositive donors, respectively) with a corresponding small increase in detection of false positives in the seronegative population.

Similarly, FIG. 5B shows FR data for seroreactive HSV-2 antigens recognized by 50% or more of the HSV-2 seropositive population. As shown, the antigens are ranked by descending frequency of recognition within the HSV-2 seropositive population. FR analysis singled out HSV-2 UL44/gC as an optimal classifier, being recognized by 100% and 0% of HSV-2 and HSV-1 seropositive donors, respectively. Other glycoproteins, notably UL1/gL and US7/gI also gave moderate discrimination.

As noted above, antigens that were revealed by T-tests to discriminate between HSV-1 and HSV-2 seropositive individuals were further subjected to ROC analysis. Table 3 shows a representative ROC analysis of data from discriminatory antigens. The ranking by AUC correlates well with ranking by BH-corrected p value. Overall, reactive antigens revealed by T tests and ROC analysis as discriminatory also showed useful discrimination in the RF analysis. For example, HSV-1 US8/gE, which was revealed by T tests and ROC to be the HSV-1 antigen that best discriminates between HSV-1 and HSV-2 seropositive donors, was recognized by 62.5% and 0% of these two populations, respectively. Similarly, HSV-2 UL44/gC, which had the most significant pBH and AUC, also showed optimal discrimination by FR analysis in the array format.

TABLE 3
HSV-1 vs. HSV-2 seropositive

| HSV-1 antigens | | | HSV-2 antigens | | |
|---|---|---|---|---|---|
| Name | AUC | pBH | name | AUC | pBH |
| UL36_S2 | 0.979 | 1.74E−08 | UL44 | 0.971 | 1.19E−12 |
| UL7 | 0.939 | 2.43E−07 | US6 | 0.968 | 1.74E−08 |
| US9 | 0.907 | 5.26E−07 | UL26.5 | 0.932 | 1.74E−08 |
| US3 | 0.746 | 7.32E−03 | UL42 | 0.932 | 3.08E−07 |
| US8* | 0.725 | 1.29E−02 | US9 | 0.929 | 4.94E−08 |
| US6 | 0.682 | 4.96E−02 | UL34 | 0.889 | 1.33E−06 |
| UL20 | 0.500 | 6.54E−03 | US11 | 0.871 | 1.49E−04 |

The identities of the reactive antigens listed in Table 1 and Table 2 show that a large number of structural proteins were reactive, notably glycoproteins such as US4/gG, US6/gD, US8/gE, UL10/gM, UL22/gH and UL44/gC. It is possible, therefore that HSV-1 and/or HSV-2 antibody response is biased towards certain types of viral proteins. To characterize this further an enrichment analysis of the HSV-1 antibody reactivity profile was performed. Each protein encoded by an HSV-1 was assigned one or more gene ontology (GO) component classifiers according to the database at www.uniprot.org. The percentage of the total number of genes assigned to each GO component present in the proteome and in the seroreactive antigens were determined, and the ratio used to determine if enrichment was evident. Analysis results in Table 4 show that virion membrane proteins account for 5.0% of the genes encoded in the HSV-1 genome, compared to 7.3% of the seroreactive antigens, indicating slight (1.5-fold) enrichment. The only other GO component that may show bias is ‘host cell nuclear membrane’. However, neither component was statistically significant (p>0.05). It is recognized, however, that enrichment may be evident in other data sets, and that such evidence of high rates of reactivity in certain protein families may provide valuable insights into HSV-1 and HSV-2 detection and treatment.

TABLE 4

| GO ID | GO Name | Total on chip | S | Fold enriched | p value |
|---|---|---|---|---|---|
| GO: 0016020 | membrane | 21 | 11 | 1.014 | 1.000 |
| GO: 0016021 | integral to membrane | 17 | 8 | 0.911 | 0.803 |
| GO: 0019012 | virion | 34 | 18 | 1.025 | 1.000 |
| GO: 0019033 | viral tegument | 16 | 10 | 1.210 | 0.443 |
| GO: 0020002 | host cell plasma membrane | 9 | 6 | 1.290 | 0.502 |
| GO: 0030430 | host cell cytoplasm | 17 | 10 | 1.139 | 0.620 |
| GO: 0033644 | host cell membrane | 15 | 7 | 0.903 | 0.792 |
| GO: 0042025 | host cell nucleus | 39 | 17 | 0.844 | 0.297 |
| GO: 0044156 | host cell junction | 2 | 1 | 0.968 | 1.000 |
| GO: 0044165 | host cell endoplasmicreticulum | 2 | 1 | 0.968 | 1.000 |
| GO: 0044174 | host cell endosome | 6 | 3 | 0.968 | 1.000 |
| GO: 0044175 | host cell endosome membrane | 6 | 3 | 0.968 | 1.000 |
| GO: 0044177 | host cell Golgi apparatus | 12 | 7 | 1.129 | 0.770 |
| GO: 0044178 | host cell Golgi membrane | 9 | 4 | 0.860 | 0.742 |
| GO: 0044196 | host cell nucleolus | 3 | 2 | 1.290 | 1.000 |
| GO: 0044200 | host cell nuclear membrane | 1 | 1 | 1.935 | 1.000 |
| GO: 0011201 | host cell nuclear inner membrane | 3 | 1 | 0.645 | 0.611 |
| GO: 0055036 | virion membrane | 12 | 9 | 1.452 | 0.139 |

| | | | | |
|---|---|---|---|---|
| Other Go component | 6 | 0 | 0.000 | 0.012 |
| No Go component | 10 | 5 | 0.968 | 1.000 |
| Total | 240 | 124 | | |

Thus, specific embodiments and applications of HSV antigen and antibody compositions and methods have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. An antigen composition comprising:
a plurality of antibody reactive antigens associated with a carrier wherein at least two of the antigens have quantified and known relative antibody reactivities with respect to sera of a population affected by HSV-2;
wherein the at least two of the antigens have a known association with a disease parameter; and
wherein the plurality of antigens are selected from the group consisting of UL1, UL3, UL5, UL6, UL7, UL10, UL14, UL17, UL18, UL23, UL26, UL26.5, UL27, UL28, UL32, UL34, UL41, UL42, UL44, UL45, UL49, UL50, UL51, UL54, US6, US7, US8, US9 and US11, or fragments thereof.

2. The antigen composition of claim 1 wherein the known reactivities are characterized by strength of reactivity.

3. The antigen composition of claim 1 wherein the known reactivities are characterized by activity state of the disease.

4. The antigen composition of claim 1 wherein the parameter is selected from the group consisting of a previous or current exposure to a pathogen, acute, latent or recurrent infection, and at least partial immunity to infection with a pathogen.

5. The antigen composition of claim 1 wherein the at least two of the antigens are present in at least 40% of a population exposed to the at least two antigens, and optionally wherein at least one of an average binding affinity and an average quantity of antibodies produced in a patient against the at least two antigens is in an upper tertile of binding affinity and quantity of antibodies produced in the patient.

6. The antigen composition of claim 1 wherein the disease parameter is previous infection with HSV, and wherein the plurality of antigens are selected from the group consisting of: UL1, UL3, UL5, UL6, UL7, UL10, UL14, UL17, UL18, UL23, UL26, UL26.5, UL27, UL28, UL32, UL34, UL41, UL42, UL44, UL45, UL49, UL50, UL51, UL54, US6, US7, US8, US9 and US11, or fragments thereof.

7. The antigen composition of claim 1 wherein the disease parameter is acute infection with HSV, and wherein the plurality of antigens are selected from the group consisting of: UL1, UL3, UL5, UL6, UL7, UL10, UL14, UL17, UL18, UL23, UL26, UL26.5, UL27, UL28, UL32, UL34, UL41, UL42, UL44, UL45, UL49, UL50, UL51, UL54, US6, US7, US8, US9, and US11, or fragments thereof.

8. The antigen composition of claim 1 wherein the disease parameter is latent infection with HSV, and wherein the plurality of antigens are selected from the group consisting of: UL1, UL3, UL5, UL6, UL7, UL10, UL14, UL17, UL18, UL23, UL26, UL26.5, UL27, UL28, UL32, UL34, UL41, UL42, UL44, UL45, UL49, UL50, UL51, UL54, US6, US7, US8, US9, and US11, or fragments thereof.

9. The antigen composition of claim 1 wherein the carrier is a pharmaceutically acceptable carrier, and wherein the composition is formulated as a vaccine.

10. The antigen composition of claim 9 wherein the vaccine comprises at least four antigens.

11. The antigen composition of claim 9 wherein the antigens or fragments thereof are recombinant.

12. The antigen composition of claim 9 wherein the antigens or fragments thereof are at least partially purified.

13. The antigen composition of claim 1 wherein the carrier is a solid carrier, and wherein the plurality of antigens is disposed on the carrier in an array.

14. The antigen composition of claim 9 wherein each of the antigens are present in a purity of greater than 60%.
